(19) United States
(12) Patent Application Publication
(10) Pub. No.: US 2012/0264989 A1
(43) Pub. Date: Oct. 18, 2012

(54) METHOD FOR DISTILLATE PRODUCTION BY MEANS OF CATALYTIC OLIGOMERIZATION OF OLEFINS IN THE PRESENCE OF METHANOL AND/OR DIMETHYL ETHER

(75) Inventors: Nikolai Nesterenko, Nivelles (BE); Delphine Minoux, Nivelles (BE); Sander Van Donk, Sainte-Adresse (FR); Jean-Pierre Dath, Beloeil Hainaut (BE)

(73) Assignee: TOTAL RAFFINAGE MARKETING, Puteaux (FR)

(21) Appl. No.: 13/501,413

(22) PCT Filed: Oct. 13, 2010
(86) PCT No.: PCT/FR10/52166

§ 371 Date: Jun. 27, 2012

(30) Foreign Application Priority Data
Oct. 13, 2009 (FR) 09 57149

(51) Int. Cl. C07C 2/02

(52) U.S. Cl. 585/520

(57) ABSTRACT

The invention relates to a process for producing distillate by oligomerization starting with a hydrocarbon-based charge in the presence of methanol and/or dimethyl ether, which may especially be of plant origin. By addition of an oxygenated compound, this process makes it possible to reduce the amounts of olefins whose chain length is too short to be exploited (typically C10, or even less) and to increase the yields of C10+ olefins.

The invention relates to a process for producing distillate by oligomerization starting with a hydrocarbon-based charge in the presence of methanol and/or dimethyl ether.

The term “distillate” means hydrocarbons containing 10 or more carbon atoms, middle distillates comprising from 10 to 20 carbon atoms and distilling in the temperature range from 145° C. to 350° C. Among the distillates, C10-C12 olefins (jet fuel) and C12+ olefins (diesel) will especially be distinguished.

Processes for the catalytic oligomerization of olefins are addition processes of olefin molecules for increasing the number of carbon atoms (or chain length) of the olefins.

The majority of the processes described in the bibliography propose solutions for oligomerizing a charge rich in olefins and more particularly in C3-C4 olefins. These olefins may be converted into oligomers under relatively mild conditions in which the contribution of the cracking reaction is negligible. The effect of the presence in the charge of a broader range of olefins in combination with an amount of inert or semi-inert material (paraffins, naphthenes and aromatics) makes the application of standard oligomerization processes less efficient.

Due to the different reactivity of C2-C10 olefins and above all due to the lower reactivity of C5-C10 olefins, it may be necessary to work at a higher temperature in order to improve the direct yield in an oligomerization process. Working at a higher temperature may lead to the cracking and aromatization of already-formed oligomers from more reactive olefins as long as part of the olefins of the charge still remains unconverted. In this case, the products of the C2-C10 olefin oligomerization processes contain large amounts of olefins bearing an excessively short chain length (less than 10 carbon atoms). These olefins cannot be used directly and must be recycled into the process in order to increase their chain length. This recycling, which may be up to 75% of the effluent produced, increases the complexity and costs of the installation. In addition, the recycling requires an additional step in the separation of olefins with a short chain length of the corresponding paraffins. The fact that these molecules have similar IPBs (initial distillation points) does not make it possible to use distillation, and necessitates the use of more sophisticated separation processes.

There is thus a need to reduce, or even eliminate, these recycling operations, by increasing the yield of distillates, especially of C10-C20 middle distillates.

The present invention provides a solution for improving the process for the oligomerization of olefinic charge containing a large amount of inert material (paraffins, naphthenes and aromatics) and olefins with different reactivity.

The invention is directed toward overcoming these drawbacks by proposing a process for the catalytic oligomerization of C3-C10 olefins which makes it possible, by adding an oxygenated compound, to reduce the amounts of olefins whose chain length is too short to be exploited (typically C10, or even less) and to increase the yields of C10+ olefins.

The invention thus allows an appreciable reduction in recycling operations, or even suppression thereof.

The presence of an oxygenated molecule as water precursor leads to moderation of the acidity of the oligomerization catalyst, limiting the cracking reaction.

On account of a lower reactivity of heavy olefins relative to light olefins in oligomerization, the presence of an oxygenated molecule as precursor for a light olefin generated progressively may lead to a higher conversion of the heavy olefins, resulting in a product whose chain length is longer.

Document U.S. Pat. No. 7,183,450 describes a process for oligomerizing a charge comprising C2-C12 olefins and oxygenated compounds, the concentration of the latter compounds in the charge being between 1000 ppm and 10% by weight. The charge contains at least 50% linear monoolefins, these linear monoolefins having a C6+ content not exceeding 20%. The oligomerization reaction is performed at a reaction temperature of from 250 to 325° C., at a pressure of from 50 bar to 500 bar, the harshest conditions being necessary when the content of oxygenated compounds is the highest.

Document WO 2007/135 053 describes a process for preparing C5 and/or C6 olefins, in which shorter-chain olefins containing from 2 to 5 carbon atoms are placed in contact with oxygenated compounds such as methanol and dimethyl ether in the presence of a zeolite-based catalyst of MTT type. The object of this document is to increase the selectivity toward C5 and/or C6, and no mention is made of increasing the yield of C10+ or C12+ olefins from a charge containing C3-C10 olefins.

Surprisingly, the Applicant has in fact discovered that, for a hydrocarbon-based charge, the addition of an amount (greater than or equal to 0.5% by weight relative to the hydrocarbon-based charge) of an oxygenated compound chosen from methanol and/or dimethyl ether (DME) makes it possible to increase the selectivity of the catalytic oligomerization process toward C10+, the degree of oligomerization being higher relative to the same charge under similar conditions, in the absence of oxygenated compounds.

To this end, a first subject of the invention concerns a process for producing distillate, C10+ hydrocarbons, by catalytic oligomerization of a hydrocarbon-based charge containing C3-C10 olefins, in which the treatment of the charge comprises at least one oligomerization step performed in at least one oligomerization reactor, in which the charge is oligomerized in the presence of at least 0.5% by weight of an oxygenated compound chosen from methanol, dimethyl ether and a methanol/dimethyl ether mixture, this (these) compound(s) possibly being, for example, of plant origin, and in which the pressure within the reactor(s) is from 1.4 to 4.9 MPa (14 to 49 bara).

The process according to the invention makes it possible especially to obtain per run products containing from 5% to 50% by weight of C12+ hydrocarbons.

Advantageously, the products obtained contain from 15% to 40% by weight of C12+ hydrocarbons.

Preferably, the products obtained contain at least 20% by weight of C12+ hydrocarbons.

Preferably, the charge contains not more than 70% by weight of oxygenated compound(s), preferably from 0.75% to 50% by weight and more particularly from 1% to 30% by weight.

When the oxygenated compound is or contains methanol, it may be obtained by fermentation of biomaterials or from synthesis gas, which may itself be obtained from renewable materials.

The use of DME as a mixture with methanol makes it possible to remove some of the heat originating from the transformation of the methanol into hydrocarbon and to use the reactor space more efficiently. In particular, the DME may be produced directly from methanol.

The hydrocarbon-based charge used may be a mixture of hydrocarbon-based effluents containing C3-C10 olefins derived from refinery or petrochemistry processes (FCC, vapor cracking, etc.). The charge may be a mixture of fractions comprising C3 FCC, C4 FCC, LCCS, LCCCS, Pygas, LCN, and mixtures, such that the content of linear olefins in the C5-fraction (C3-C5 hydrocarbons) relative to the total C3-C10 charge is less than or equal to 40% by weight.

The total olefin content in the C5-(C3-05) fraction relative to the total C3-C10 charge supplied for the oligomerization may be greater than 40% by weight if the isoolefins are present in an amount of at least 0.5% by weight.

The total content of linear olefins may be greater than 40% by weight relative to the total charge of C3-C10 if the linear C6+ olefins (C6, C7, C8, C9, C10) are present in an amount of at least 0.5% by weight.

This charge may especially contain olefins, paraffins and aromatic compounds in all proportions, in conformity with the rules described above.

The process according to the invention may be performed without prior separation of the heaviest hydrocarbons of the hydrocarbon-based charge.

The hydrocarbon-based charge will preferably contain a small amount of dienes and of acetylenic hydrocarbons, especially less than 100 ppm of diene, preferably less than 10 ppm of C3-C5 dienes. To this end, the hydrocarbon-based charge will be treated, for example, by selective hydrogenation optionally combined with adsorption techniques.

The hydrocarbon-based charge will preferably contain a small amount of metals, for example less than 50 ppm and preferably less than 10 ppm. To this end, the hydrocarbon-based charge will be treated, for example, by selective hydrogenation optionally combined with adsorption techniques.

Advantageously, the hydrocarbon-based charge used has undergone a partial extraction of the isoolefins it contains, for example by treatment in an etherification unit, thus allowing concentration as linear olefins.

In general, commercially available olefinic charges bring about deactivation of the oligomerization catalyst that is faster than expected. Although the reasons for such a deactivation are not clearly understood, it is considered that the presence of certain sulfur compounds is at least partly responsible for this drop in activity and selectivity. In particular, it would appear that aliphatic thiols, sulfides and disulfides of low molecular weight are more particularly troublesome.

It is thus established that the acceptable sulfur content in a charge of an oligomerization process must be low enough for the activity of the catalyst used not to be inhibited. In general, the sulfur content is less than or equal to 100 ppm, preferably less than or equal to 10 ppm, or even less than or equal to 1 ppm.

The removal of these sulfur compounds requires hydrotreatment steps that increase the total cost of the process, and which may lead to a reduction in the amount of olefins. This loss may prove to be very penalizing for C5-C10 fractions typically containing from 200 to 400 ppm of sulfur.

There is thus also a need to develop an oligomerization process that allows the treatment of commercially available olefinic charges without a severe prior hydrotreatment.

It is common practice to add water to the charge of a catalytic oligomerization process. This addition of water makes it possible especially to control the temperature of the oligomerization reactor, in particular during the startup of the reactor, when the catalyst is fresh and the exothermicity is greatest. The presence of water-precursor oxygenated compounds in the charge used for the process according to the invention has the advantage of increasing the sulfur tolerance of the oligomerization catalysts. The lifetime of the catalyst may thus be increased. As a result of the contents of oxygenated compounds used, the water formed during the oligomerization represents more than 0.25% by weight of the hydrocarbon-based charge.

By way of example, in order to prevent the catalytic activity of the catalyst from being substantially inhibited, the nitrogen content of the hydrocarbon-based charge is not greater than 1 ppm by weight (calculated on an atomic basis), preferably not greater than 0.5 ppm and more preferably 0.3 ppm. Furthermore, by way of example, the chloride content of the hydrocarbon-based charge is not greater than 0.5 ppm by weight (calculated on an atomic basis), preferably not greater than 0.4 ppm and more preferably 0.1 ppm.

To this end, the hydrocarbon-based charge used may have undergone a prior treatment, for example a partial hydrotreatment, a selective hydrogenation and/or a selective adsorption.

The effluents of the oligomerization process will then be conveyed to a separation zone, in order to separate, for example, the fractions into an aqueous fraction, C5-C9 (gasoline), C10-C12 (jet fuel) and C12+ (diesel). The fractions C5-C9, C10-C12 and C12+ may undergo drying.

Thus, the invention makes it possible especially to obtain a jet fuel (C10-C12) from alcohols of plant origin.

The fractions C10-C12 and C12+ separated from the effluents of the oligomerization process may undergo a hydrogenation in order to saturate the olefinic compounds and to hydrogenate the aromatic compounds. The product obtained has a high cetane number and excellent properties for use as a fuel of jet or diesel type, or the like.

In one embodiment, the effluent derived from the charge oligomerization step is conveyed into the separation zone, in which the C2-C4 olefins are also separated out and in which part of the C2-C4 olefins may be recycled as charge for the step of oligomerization of the hydrocarbon-based charge, the rest of the C2-C4 olefins being purged, for example.

In another embodiment, the hydrocarbon-based charge is oligomerized in two oligomerization reactors in series, the effluent leaving the second reactor being conveyed into the separation zone in which the C2-C4 olefins are also separated out and in which part of the C2-C4 olefins may be recycled as charge for the step of oligomerization of the hydrocarbon-based charge, the rest of the C2-C4 olefins being purged, for example.

As a variant, the oxygenated compound may be introduced at the inlet or into the second oligomerization reactor.

Without wishing to be bound by theory, it would appear that, under the oligomerization conditions, methanol, DME or a methanol/DME mixture reacts with the olefins by adding a carbon to the olefin chain. The chain length of the olefins would thus be extended by one carbon at a time on each reaction with methanol and/or DME. Since these oxygenated compounds have high activity, this reaction with olefins would be faster than the oligomerization reaction between two olefins.

Moreover, the presence of oxygenated compounds in the hydrocarbon-based charge of the oligomerization process increases the partial pressure of olefins, which makes it possible to improve the yield for the oligomerization process.

The process according to the invention may be performed under the conditions described below.

Advantageously, the hydrocarbon-based charge is oligomerized by being placed in contact with an acidic catalyst in the presence of a reducing compound, such as hydrogen. In particular, the presence of a reducing atmosphere, and possibly of water, improves the stability of the catalyst used.

The process according to the invention has the advantage of being able to be performed in an existing installation.

For example, an installation containing several reactors may be used, in which the exothermicity of the reaction may be controlled so as to avoid excessive temperatures. Preferably, the maximum temperature difference within the same reactor will not exceed 75° C.

The reactor(s) may be of the isothermal or adiabatic type with a fixed or moving bed. The oligomerization reaction may be performed continuously in one configuration comprising a series of fixed-bed reactors mounted in parallel, in which, when one or more reactors are in service, the other reactors undergo regeneration of the catalyst.

The process may be performed in one or more reactors.

Preferably, the process will be performed using two separate reactors.

The reaction conditions for the first reactor will be chosen so as to convert part of the olefinic compounds with a low carbon number (C3-C5) into intermediate olefins (C6+).

Advantageously, the first reactor will comprise a first catalytic zone and will function at high temperature, for example greater than or equal to 250° C., and at moderate pressure, for example less than 50 bar.

The second reactor will preferably operate at temperatures and pressures chosen so as to promote the oligomerization of heavy olefins to distillate. The effluent from the first reactor, comprising the unreacted olefins, the intermediate olefins, water and possibly other compounds such as paraffins and possibly a reducing gas, then undergoes an oligomerization in this second reactor comprising a second catalytic zone, which makes it possible to obtain an effluent of heavier hydrocarbons, rich in distillate.

Advantageously, the first reactor will function at a lower pressure and at a higher temperature and hourly space velocity than the second reactor.

It may optionally be envisioned to use the pressure difference between the two reactors in order to perform a flash separation step. Thus, for example, in the case where the oxygenated compound is ethanol, the unreacted ethylene and the other light gases may be readily separated out and removed from the heavier hydrocarbons forming the liquid phase. An excess of water may then optionally be removed.

For example, when the hydrocarbon-based charge is oligomerized in an installation comprising several reactors in series, the presence of oxygenated compounds is not obligatory at the inlet of the first reactor. The oxygenated compounds may be injected into the middle of the first reactor or into the inlet of the second reactor, for example. It is important for the total amount of an added oxygenated compound to be greater than or equal to 0.5% by weight relative to the hydrocarbon-based charge.

All of the oxygenated compound may be added to the charge before it enters the oligomerization reactor(s), or partly before it enters the oligomerization reactor(s), the remaining part being added to the oligomerization reactor(s), for example as a quench.

The mass throughput through the oligomerization reactor(s) will advantageously be sufficient to enable a relatively high conversion, without being too low, so as to avoid adverse side reactions. The hourly space velocity (weight hourly space velocity, WHSV) of the charge will be, for example, from 0.1 to 20 h⁻¹, preferably from 0.5 to 10 h⁻¹ and more preferably from 1 to 8 h⁻¹.

The temperature at the reactor inlet will advantageously be sufficient to allow a relatively high conversion, without being very high, so as to avoid adverse side reactions. The temperature at the reactor inlet will be, for example, from 150° C. to 400° C., preferably 200-350° C. and more preferably from 220 to 350° C.

The pressure across the oligomerization reactor(s) will advantageously be sufficient to allow a relatively high conversion, without being too low, so as to avoid adverse side reactions. The pressure across the reactor will be, for example, from 8 to 500 bara, preferably 10-150 bara and more preferably from 14 to 49 bara (bar, absolute pressure).

As regards the nature of the catalyst, a first family of catalysts comprises an acidic catalyst either of amorphous or crystalline aluminosilicate type, or a silicoaluminophosphate, in H+ form, chosen from the following list and optionally containing alkali metals or alkaline-earth metals:

MFI (ZSM-5, silicalite-1, boralite C, TS-1), MEL (ZSM-11, silicalite-2, boralite D, TS-2, SSZ-46), ASA (amorphous silica-alumina), MSA (mesoporous silica-alumina), FER (Ferrierite, FU-9, ZSM-35), MIT (ZSM-23), MWW (MCM-22, PSH-3, ITQ-1, MCM-49), TON (ZSM-22, Theta-1, NU-10), EUO (ZSM-50, EU-1), ZSM-48, MFS (ZSM-57), MTW, MAZ, SAPO-11, SAPO-5, FAU, LTL, BETA MOR, SAPO-40, SAPO-37, SAPO-41 and the family of microporous materials composed of silica, aluminum, oxygen and possibly boron.

Zeolite may be subjected to various treatments before use, which may be: ion exchange, modification with metals, steam treatment (steaming), acid treatments or any other dealumination method, surface passivation by deposition of silica, or any combination of the abovementioned treatments.

The content of alkali metals or rare-earth metals is from 0.05% to 10% by weight and preferentially from 0.2% to 5% by weight. Preferentially, the metals used are Mg, Ca, Ba, Sr, La and Ce, which are used alone or as a mixture.

A second family of catalysts used comprises phosphate-modified zeolites optionally containing an alkali metal or a rare-earth metal. In this case, the zeolite may be chosen from the following list:

MFI (ZSM-5, silicalite-1, boralite C, TS-1), MEL (ZSM-11, silicalite-2, boralite D, TS-2, SSZ-46), ASA (amorphous silica-alumina), MSA (mesoporous silica-alumina), FER (Ferrierite, FU-9, ZSM-35), MIT (ZSM-23), MWW (MCM-22, PSH-3, ITQ-1, MCM-49), TON (ZSM-22, Theta-1, NU-10), EUO (ZSM-50, EU-1), MFS (ZSM-57), ZSM-48, MTW, MAZ, FAU, LTL, BETA MOR.

The zeolite may be subjected to various treatments before use, which may be: ion exchange, modification with metals, steam treatment (steaming), acid treatments or any other dealumination method, surface passivation by deposition of silica, or any combination of the abovementioned treatments.

The content of alkali metals or of rare-earth metals is from 0.05% to 10% by weight and preferentially from 0.2% to 5% by weight. Preferentially, the metals used are Mg, Ca, Ba, Sr, La and Ce, which are used alone or as a mixture.

A third family of catalysts used comprises difunctional catalysts, comprising:
a support, from the following list: MFI (ZSM-5, silicalite-1, boralite C, TS-1), MEL (ZSM-11, silicalite-2, boralite D, TS-2, SSZ-46), ASA (amorphous silica-alumina), MSA (mesoporous silica-alumina), FER (Ferrierite, FU-9, ZSM-35), MIT (ZSM-23), MWW (MCM-22, PSH-3, ITQ-1, MCM-49), TON (ZSM-22, Theta-1, NU-10), EUO (ZSM-50, EU-1), MFS (ZSM-57), ZSM-48, MTW, MAZ, BETA, FAU, LTL, MOR, and microporous materials of the family ZSM-48 consisting of silicon, aluminum, oxygen and optionally boron. MFI or MEL (Si/Al>25), MCM-41, MCM-48, SBA-15, SBA-16, SiO2, Al2O3, hydrotalcite, or a mixture thereof;
a metallic phase (Me) to a proportion of 0.1% by weight, the metal being selected from the following elements: Zn, Mn, Co, Ni, Ga, Fe, Ti, Zr, Ge, Sn and Cr used alone or as a mixture. These metal atoms may be inserted into the tetrahedral structure of the support via the tetrahedral unit [MeO₂]. The incorporation of this metal may be performed either by adding this metal during the synthesis of the support, or it may be incorporated after synthesis by ion exchange or impregnation, the metals then being incorporated in the form of cations, and not integrated into the structure of the support.

The zeolite may be subjected to various treatments before use, which may be: ion exchange, modification with metals, steam treatment (steaming), acid treatments or any other dealumination method, surface passivation by deposition of silica, or any combination of the abovementioned treatments.

The content of alkali metals or of rare-earth metals is from 0.05% to 10% by weight and preferentially from 0.2% to 5% by weight. Preferentially, the metals used are Mg, Ca, Ba, Sr, La and Ce, used alone or as a mixture.

The catalyst may be a mixture of the materials described previously in the three families of catalyst. In addition, the active phases may themselves also be combined with other constituents (binder, matrix) giving the final catalyst increased mechanical strength, or improved activity.

If the hydrocarbon-based charge is oligomerized in an installation comprising several reactors in series, the reactors of the series may be charged with the same catalyst or a different one.

The invention is now described with reference to the examples and the attached drawings, which are not limiting, in which:

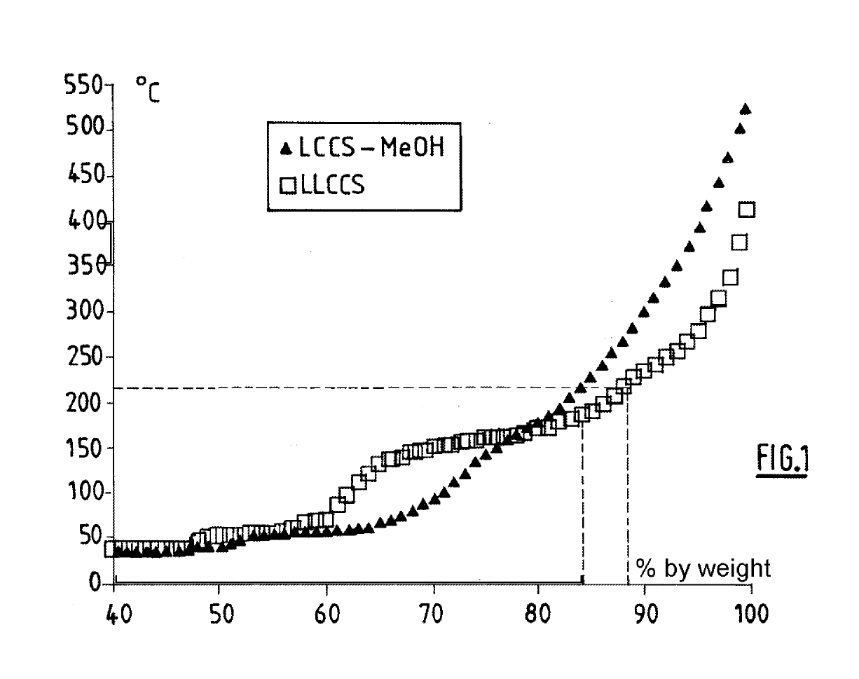

FIG. 1 represents the curves of simulated distillation of the organic phases of the product obtained by means of the process according to the invention, and in the absence of oxygenated compounds, under the conditions of Examples 15 and 16;

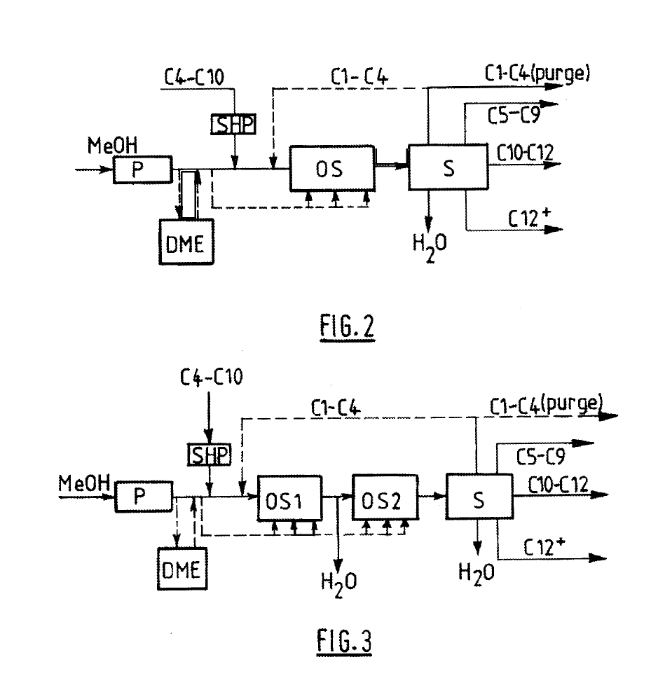

FIGS. 2 to 4 schematically represent various embodiments of the process according to the invention.

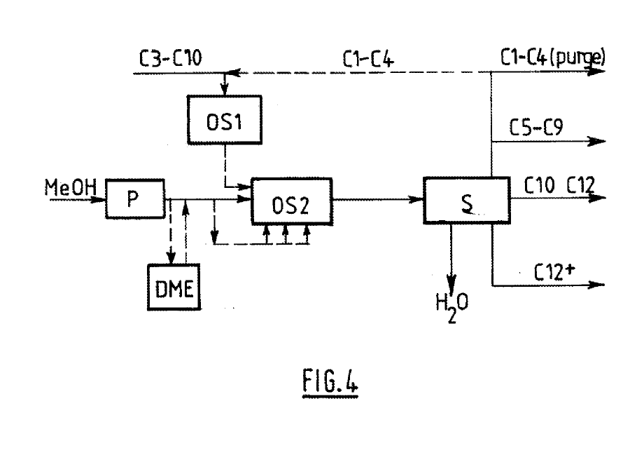

In each of the FIGS. 2 to 4:
OS represents an oligomerization zone, FIGS. 3 and 4 comprising two oligomerization zones, OS1 et OS2,
S represents a separation zone,
SHP represents a zone of selective hydrogenation and/or of selective adsorption,
DME is a reactor for the production of dimethyl ether from the oxygenated compounds,
P represents a zone for purification of the oxygenated compound.

On these FIGS. 2-4, the dashed lines represent process options.

Each oligomerization zone represents, for example, an oligomerization reactor.

Naturally, these embodiments may also be operated with DME alone.

The scheme represented in FIG. 2 corresponds to a process in which the charge consisting of C4-C10 hydrocarbon-based compounds is mixed, after selective hydrogenation (SHP), with methanol, optionally comprising DME, and then treated in an oligomerization zone OS. The effluent leaving this zone OS is conveyed into the separation zone S.

In this zone S, the water is removed and the olefins are separated into C2-C4, C5-C9 (gasoline), C10-C12 (jet) and diesel (C12+). Part of the light C2-C4 olefins may optionally be recycled as charge for the oligomerization zone OS.

All the oxygenated compounds (methanol optionally comprising DME) may be added at the inlet of the zone OS or inside this zone (dashed lines).

The process represented schematically in FIG. 3 comprises two oligomerization zones OS1 and OS2.

The charge consisting of C4-C10 hydrocarbon-based compounds is mixed, after selective hydrogenation (SHP), with methanol (and optionally DME), and then treated in a first oligomerization zone OS1. The effluent leaving this zone OS1 is conveyed to the second oligomerization zone OS2. The effluent leaving zone OS2 is conveyed to the separation zone S.

In this zone S, the water is removed and the olefins are separated into C2-C4, C5-C9 (gasoline), C10-C12 (jet fuel) and diesel (C12+). Part of the light C2-C4 olefins thus separated out may optionally be returned as charge for the first oligomerization zone OS1.

All the oxygenated compounds (methanol optionally mixed with DME) may be added at the inlet of zone OS1 or inside the two zones OS1 and OS2 (dashed lines). In addition, depending on the operating conditions of these two zones OS1 and OS2, removal of water may be performed between the two zones OS1 and OS2.

The process shown schematically in FIG. 4 comprises two oligomerization zones OS1 and OS2.

The charge consisting of C3-C10 hydrocarbon-based compounds undergoes a first oligomerization in zone OS1. The effluent leaving the first zone OS1 is mixed with methanol (and optionally DME) and then treated in a second oligomerization zone OS2. The effluent leaving this second zone OS2 is conveyed to the separation zone S.

In this zone S, the water is removed and the olefins are separated into C2-C4, C5-C9 (gasoline), C10-C12 (jet fuel) and diesel (C12+). Part of the light C2-C4 olefins thus separated out may optionally be returned as charge for the first oligomerization zone OS1.

All the methanol (optionally mixed with DME) may be added at the inlet of zone OS2 or inside this zone (dashed lines).

### EXAMPLES

### Example 1

### Preparation of Catalyst A

A commercial sample of silicalite (MFI, Si/Al=200) in NH₄ form (ammonium) was calcined at 550° C. for 6 hours in order to convert it into H (proton) form. The product thus obtained is named catalyst A.

### Example 2

### Preparation of Catalyst B

A sample of SAPO-11 was prepared according to document U.S. Pat. No. 4,310,440.

A reaction mixture, whose molar composition is given in Table 1, was prepared in a Teflon vessel, and stirred until homogeneous for about 30 minutes at room temperature. The Teflon vessel was then placed in a 200 ml stainless-steel autoclave. After closure, this autoclave is maintained at high temperature (200° C.) with stirring for 24 hours. After cooling to room temperature, the solid is separated from the liquid phase by centrifugation and then dried at 110° C. overnight and calcined under a stream of air at 550° C. for 6 hours, the calcination making it possible to remove the organic templates. The product thus obtained is named catalyst B.

TABLE 1
mole ratio of the starting reaction mixture

| Dipropylamine | Al₂O₃ | P₂O₅ | SiO₂ | H₂O |
|---|---|---|---|---|
| 2 | 1 | 1 | 0.05 | 50 |

### Example 3

### Preparation of Catalyst C

A sample of SAPO-5 was synthesized according to Stud. Surf. Sci. Catal., 84, 1994, 867-874.

The reaction mixture, the molar composition of which is given in Table 2, was prepared in a Teflon vessel and stirred until homogeneous for about 30 minutes at room temperature. The Teflon vessel was then placed in a 200 ml stainless-steel autoclave. After closure, this autoclave is maintained at high temperature (200° C.) with stirring for 7 days. After cooling to room temperature, the solid is separated from the liquid phase by centrifugation, and then dried at 110° C. overnight and calcined in air at 550° C. for 6 hours, the calcination making it possible to remove the organic template. The product thus obtained is named catalyst C.

TABLE 2
mole ratio of the starting reaction mixture

| TPAOH | TPABr | Al₂O₃ | P₂O₅ | SiO₂ | EG |
|---|---|---|---|---|---|
| 1 | 1 | 1 | 1 | 0.4 | 12 |

### Example 4

### Preparation of Catalyst D

A sample of SAPO-37 was prepared according to document U.S. Pat. No. 4,440,871.

A reaction mixture, the molar composition of which is given in Table 3, was prepared in a Teflon vessel and stirred until homogeneous for about 30 minutes at room temperature. The Teflon vessel was then placed in a 200 ml stainless steel autoclave. After closure, this autoclave was maintained at high temperature (200° C.) with stirring for 28 hours. After cooling to room temperature, the solid is separated from the liquid phase by centrifugation and then dried at 110° C. overnight and calcined under a stream of air at 550° C. for 6 hours, the calcination making it possible to remove the organic template. The product thus obtained is named catalyst D.

TABLE 3
mole ratio of the starting reaction mixture

| (TPA)₂O | (TMA)₂O | Al₂O₃ | P₂O₅ | SiO₂ | H₂O |
|---|---|---|---|---|---|
| 1 | 0.025 | 1 | 1 | 0.4 | 50 |

### Example 5

### Preparation of Catalyst E

A sample of SAPO-41 was prepared according to document U.S. Pat. No. 4,440,871.

A reaction mixture, the molar composition of which is given in Table 4, was prepared in a Teflon vessel and stirred until homogeneous for about 30 minutes at room temperature. The Teflon vessel was then placed in a 200 ml stainless steel autoclave. After closure, this autoclave was maintained at high temperature (200° C.) with stirring for 24 hours. After cooling to room temperature, the solid is separated from the liquid phase by centrifugation and then dried at 110° C. overnight and calcined under a stream of air at 550° C. for 6 hours, the calcination making it possible to remove the organic template. The product thus obtained is named catalyst E.

TABLE 4
mole ratio of the starting reaction mixture

| Dipropylamine | Al₂O₃ | P₂O₅ | SiO₂ | H₂O |
|---|---|---|---|---|
| 3 | 0.85 | 1 | 0.1 | 50 |

### Example 6

### Preparation of Catalyst G

MFI, Si/Al=40 with a crystal size of 0.1-0.3 μm supplied by Zeolyst Int. in NH₄ form was calcined at 550° C. for 6 hours in order to convert it into H (proton) form. The product thus obtained is named catalyst G.

### Examples 7-14

Oligomerization tests were performed using the catalysts prepared in Examples 1 to 6.

The catalysts in the form of grains (35-45 mesh) are charged into a fixed-bed tubular reactor. Before the tests, the catalysts are activated at 550° C. under a stream of nitrogen for 6 hours.

The hydrocarbon-based charge used for the oligomerization tests is a mixture of n-pentane and 1-hexene. The tested oxygenated compound is methanol.

The hydrocarbon-based charge mixed with the methanol was placed in contact with catalyst A under the following conditions:

reactor inlet temperature: 300° C.

pressure P: 15 barg (˜1.5 MPa)

hourly space velocity: 4 h⁻¹

(P (barg)=P bar−Patm (˜1 bar))

Analysis of the products obtained was performed online by gas chromatography, the chromatograph being equipped with a capillary column.

On leaving the reactor, the gaseous phase, the liquid organic phase and the aqueous phase were separated. No recycling was performed.

The results of the tests are given in Table 5 “on dry & coke free basis”.

Methanol was considered as being an olefin (—CH2-).

In Examples 7 to 10, a variable amount of methanol was used as a mixture with the charge described above, in the presence of catalyst A of Example 1. The corresponding results are collated in Table 5.

In Examples 11 to 14, several of the catalysts prepared in Examples 1 to 6 were used for the oligomerization of the same charge composed of 50% methanol +20% 1-hexene +30% n-pentane. The corresponding results are collated in Table 6.

The results collated in Table 5 demonstrate the beneficial effect of the presence of methanol in the charge. The addition of methanol clearly makes it possible to increase the yields of heavy fractions (especially of C12+) and to reduce the yields of light fractions (C1-C5) in the presence of paraffins.

The results collated in Table 5 illustrate the positive effect of injecting methanol for increasing the yield of C12+ and for reducing the yield of cracking products (C1-C5).

The results collated in Table 6 illustrate the performance of various catalysts of silicoaluminophosphate type in oligomerization. The most efficient catalysts in terms of increasing the yield of heavy fractions (especially of C12+) and of reducing the yields of light fractions (C1-C5) are catalysts A and C.

TABLE 5

| | Examples | | | |
|---|---|---|---|---|
| | 7 | | | |
| | (comparative) | 8 | 9 | 10 |
| Catalyst | A | | | |
| Charge | 70% 1- | 10% | 30% | 50% |
| | hexene | methanol | methanol | methanol |
| | 30% n- | 60% 1- | 40% 1- | 20% 1- |
| | pentane | hexene | hexene | hexene |
| | | 30% n- | 30% n- | 30% n- |
| | | pentane | pentane | pentane |
| TOS, h | 5 | | | |
| WHSV, h⁻¹ | 4 | | | |
| P, barg | 15 | | | |
| T, ° C. | 300 | | | |
| Olefin yield, | | | | |
| mass % | | | | |
| C1-C5 (+DME) | 17.8 | 12.1 | 11.9 | 13.9 |
| C6-C11 | 42.6 | 44.6 | 34.2 | 40.5 |
| (comprising 1- | | | | |
| hexene) | | | | |
| 1-hexene | 23.3 | 20 | 30.3 | 19.8 |
| C12+ | 16.3 | 23.3 | 23.6 | 25.8 |

TABLE 6

| Examples | 11 | 12 | 13 | 14 | 10 |
|---|---|---|---|---|---|
| Catalyst | B | C | D | E | A |

| Charge | 50% methanol + 20% 1-hexene + 30% n-pentane |
|---|---|
| TOS, h | 5 |
| WHSV, h⁻¹ | 4 |
| P, barg | 15 |
| T, ° C. | 300 |

Olefin yield, mass %

| | | | | | |
|---|---|---|---|---|---|
| C1-C5 (+DME) | 1.1 | 6.2 | 1.35 | 1.3 | 13.9 |
| C6-C11 | 67.7 | 31.6 | 8.1 | 1.6 | 40.5 |
| (comprising 1- | | | | | |
| hexene) | | | | | |
| 1-hexene | 24 | 26.5 | 87.35 | 17.4 | 19.8 |
| C12+ | 7.2 | 35.7 | 3.2 | 19.7 | 25.8 |

### Example 15

An oligomerization test was performed on 6.5 g of catalyst G as grains (35-45 mesh) charged into a fixed-bed tubular reactor. Before the tests, the catalyst was activated at 550° C. under a stream of nitrogen for 6 hours. After activation, the reactor was cooled to the reaction temperature and the catalyst was placed in contact with the charge.

The hydrocarbon-based charge used for this oligomerization test is an LLCCS (light LCCS, IPB-60) containing 83% by weight of C5 hydrocarbons (of which 59% by weight are olefins and 41% by weight are paraffins) and 10 ppm of sulfur. The content of linear olefins in the C5- fractions was 27.2% by weight.

The tested oxygenated compound is methanol.

75% by weight of LLCCS and 25% by weight of methanol were placed in contact with catalyst G under the following conditions:

reactor inlet temperature: 260° C.

pressure P: 15 barg (˜1.5 MPa)

hourly space velocity: 2 h⁻¹

On leaving the reactor, the gaseous phase, the liquid organic phase and the aqueous phase were separated. No recycling was performed.

The curve of simulated distillation measured for the liquid organic phase is given in FIG. 1.

### Example 16

### Comparative

The same test was performed under the same conditions as for Example 15, but in the absence of methanol. The simulated distillation curve is given in FIG. 1.

The results of FIG. 1 show that the transformation of LLCCS into heavy hydrocarbons (C12+) in the presence of MeOH leads to higher yields.

1. A process for producing distillate, C10+ hydrocarbons, by catalytic oligomerization of a hydrocarbon-based charge containing C3-C10 olefins, in which the treatment of the charge comprises at least one oligomerization step performed in at least one oligomerization reactor, in which the charge is oligomerized in the presence of at least 0.5% by weight of an oxygenated compound chosen from methanol, dimethyl ether and a methanol/dimethyl ether mixture, this (these) compound(s) possibly being of plant origin, and in which the pressure within the reactor(s) is from 1.4 to 4.9 MPa.

2. The process as claimed in claim 1, in which the hydrocarbon-based charge is oligomerized in the presence of at most 70% by weight of oxygenated compound, preferably from 0.75% to 50% by weight and more particularly from 1% to 30% by weight.

3. The process as claimed in claim 1, in which the effluent derived from the charge oligomerization step is conveyed to a separation zone in which at least the C2-C4 olefins are separated out and in which part of the C2-C4 olefins is recycled as charge for the oligomerization step with the hydrocarbon-based charge, the rest of the C2-C4 olefins being purged, for example.

4. The process as claimed in claim 1, in which the hydrocarbon-based charge is oligomerized in two oligomerization reactors in series, the effluent leaving the second reactor being conveyed into a separation zone in which at least the C2-C4 olefins are separated out and in which part of the C2-C4 olefins is recycled as charge for the step of oligomerization of the hydrocarbon-based charge, the rest of the C2-C4 olefins being purged, for example.

5. The process as claimed in claim 1, in which at least part of the oxygenated compound is added to the hydrocarbon-based charge before it enters the oligomerization reactor(s).

6. The process as claimed in claim 1, in which at least part of the oxygenated compound is added to an oligomerization reactor, and/or between two oligomerization reactors.

7. The process as claimed in claim 1, in which the hourly space velocity of the hydrocarbon-based charge is from 0.1 to 20 h⁻¹, preferably from 0.5 to 10 h⁻¹ and more preferably from 1 to 8 h⁻¹.

8. The process as claimed in claim 1, in which the inlet reaction temperature of the reactor(s) is from 150 to 400° C., preferably 200-350° C. and more preferably from 220 to 350° C.
